(19) United States
(12) Patent Application Publication
(10) Pub. No.: US 2023/0181032 A1
(43) Pub. Date: Jun. 15, 2023

(54) MEASUREMENTS OF KERATOMETRY AND AXIAL LENGTH

(71) Applicant: Welch Allyn, Inc., Skaneateles Falls, NY (US)

(72) Inventors: David L. Kellner, Baldwinsville, NY (US); Vivian Loomis Hunter, Auburn, NY (US); Yaolong Lou, Loyang (SG); Zhang Yi, Wuhan (CN)

(21) Appl. No.: 18/079,581

(22) Filed: Dec. 12, 2022

Related U.S. Application Data
(60) Provisional application No. 63/289,041, filed on Dec. 13, 2021.

(51) Int. Cl. A61B 3/107; A61B 3/00; A61B 3/14

(52) U.S. Cl. CPC A61B 3/107; A61B 3/14; A61B 3/0025; A61B 3/0041

(57) ABSTRACT

A vision screening device captures image(s) of reflected radiation from light source(s) and uses the images to determine a cornea curvature. The light source(s) may comprise infrared LEDs and be configured in a predetermined pattern. Based on the cornea curvature, an axial length is determined and/or a prescription for a patient is determined. Other characteristics of the patient may be used to determine axial length. Based on the axial length, a recommendation for the patient is generated and displayed.

### CROSS REFERENCE TO RELATED APPLICATION

This application is a Nonprovisional of, and claims priority to, U.S. Provisional Pat. Application No. 63/289,041, filed Dec. 13, 2021, the entire disclosure of which is incorporated herein by reference.

### TECHNICAL FIELD

The present application relates to systems and methods for measuring keratometry and axial length with a vision screening device. More particularly, this disclosure relates to systems and methods for determining keratometry through photorefraction.

### BACKGROUND

Visual acuity is a person’s ability to identify characters at a particular distance. “Normal” visual acuity is generally determined during a vision screening exam and is generally defined as being 20/20 vision. However, various conditions impact whether a person has “normal” vision, such as whether the person has an astigmatism in one or both eyes and/or whether a person has myopia (e.g., is nearsighted). Myopia can develop gradually or rapidly, tends to run in families, and results in faraway objects appearing blurry. Astigmatism occurs when either the front surface of the eye (cornea) or the lens inside the eye has mismatched curves and results in blurred vision and/or myopia. Treatment options for both astigmatism and myopia include eyeglasses, contact lenses, and surgery such as LASIK.

During a vision screening exam, a person without “normal” vision may require various additional tests and/or measurements to be performed. Each additional test and/or measurement can require additional equipment in order to be performed and increases the time the vision screening exam lasts. One such measurement performed is keratometry (e.g., measurement of a curvature of the cornea). Cornea curvature determines the power of the cornea and differences in power across the cornea (opposite meridians) results in astigmatism. Accordingly, keratometry is used to assess an amount of astigmatism a person may have. Additionally, keratometry is used to fit contacts with the person. Traditionally, keratometry is measured manually and with methods that require the use of prisms (e.g., via a fixed object size with variable image size (e.g., variable doubling) and/or via a fixed image size with variable object size (fixed doubling)). Keratometry can also be measured using automated methods (e.g., auto-keratometry). Auto-keratometry methods utilize illuminated target mires and focus the reflected image on electrical photosensitive devices. While auto-keratometry devices are more compact and less time-consuming, portability is poor. Another measurement of the eye that is performed is axial length. Axial length is strongly correlated with myopia and tracking the progression of myopia. Traditional methods for measuring axial length include ultrasonic measurement, partial coherence interferometry (PCI), silicone oil filled eye axis measurement, and/or photographic measurements. However, existing methods are highly complex, require the use of expensive equipment, have poor portability. Additionally, some techniques for measuring axial length are less accurate than others, such as using existing photographic measurement techniques.

In some instances, a large number of people undergo visual acuity screening in a given time frame. For example, a group of kindergarten students at a public school may be screened during a class period. Usually, each kindergarten student waits their turn to be screened, then each student reads up to 30 characters for each eye. This is a time-consuming undertaking, which can test the limits of the children’s patience. In some examples, a hand-held device is used during the vision screening exams to determine visual acuity, such as via eccentric photorefraction. However, current hand-held devices do not measure the keratometry of a person’s eye or an axial length. Additionally, as some countries require keratometry and axial length measurements as part of vision screening exams, current handheld devices are insufficient. Accordingly, measuring keratometry and/or axial length can be time consuming, costly (e.g., such as requiring additional equipment), and inefficient (e.g., such as for groups).

### SUMMARY

In an example of the present disclosure, a system comprises a processing unit, one or more light sources operatively connected to the processing unit, a light sensor operatively connected to the processing unit, and non-transitory computer-readable media. The non-transitory computer-readable media can store instructions that, when executed by the processing unit, cause the processing unit to perform operations comprising causing the one or more light sources to direct radiation to a cornea of a patient in a predetermined pattern, causing the light sensor to capture a portion of the radiation that is reflected from the cornea of the patient, generating an image based on the portion of the radiation, the image illustrating a dot indicative of reflected radiation, determining a location within the image, the location being associated with the dot indicative of the reflected radiation, determining a difference between the location of the returned radiation and an expected location within an expected return image, the expected location being associated with where the dot indicative of the reflected radiation is expected to be captured, and determining, based at least in part on the difference, a curvature of the cornea.

In yet another example of the present disclosure, an example vision screening device includes a processing unit, a housing, one or more light sources disposed within the housing and operatively connected to the processing unit, a light sensor disposed within the housing and operatively connected to the processing unit, and memory. The memory may store instructions that, when executed by the processing unit, cause the vision screening device to: cause the one or more light sources to direct radiation to a cornea of a patient in a predetermined pattern, cause the light sensor to capture a portion of the radiation that is reflected from the cornea of the patient, generate an image based on the portion of the radiation, the image illustrating a dot indicative of reflected radiation, determine a location within the image, the location being associated with the dot indicative of the reflected radiation, determine a difference between the location of the returned radiation and an expected location within an expected return image, the expected location being associated with where the dot indicative of the reflected radiation is expected to be captured, and determine, based at least partly on the difference, a curvature of the cornea.

In another example of the present disclosure, a system comprises a processing unit, one or more light sources operatively connected to the processing unit, a light sensor operatively connected to the processing unit, and one or more non-transitory computer-readable media storing instructions. The instructions, when executed by the processing unit, cause the processing unit to perform operations comprising: cause the one or more light sources to direct radiation to a first cornea of an eye of a patient, cause the light sensor to capture an image of returned radiation that is reflected from the first cornea of the patient, determine, based at least partly on the image, a curvature of the first cornea, determine, based at least partly on the curvature of the first cornea, an axial length associated with the eye, and generate, based at least partly on the axial length, a recommendation associated with the patient.

In yet another example of the present disclosure, an example vision screening device includes a housing, a processing unit disposed within the housing, one or more light sources disposed within the housing and operatively connected to the processing unit, a light sensor disposed within the housing and operatively connected to the processing unit, and memory. The memory may store instructions that, when executed by the processing unit, cause the vision screening device to: cause the one or more light sources to direct radiation to a first cornea of an eye of a patient, cause the light sensor to capture an image of returned radiation that is reflected from the first cornea of the patient, determine, based at least partly on the image, a curvature of the first cornea, determine, based at least partly on the curvature of the first cornea, an axial length associated with the eye, and generate, based at least partly on the axial length, a recommendation associated with the patient

The details of one or more embodiments are set forth in the accompanying drawings and the description below. Other features, objects, and advantages of these embodiments will be apparent from the description, drawings, and claims.

### DESCRIPTION OF THE FIGURES

The present invention may comprise one or more of the features recited in the appended claims and/or one or more of the following features or combinations thereof. Additionally, in this specification and drawings, features similar to or the same as features already described may be identified by reference characters or numerals which are the same as or similar to those previously used. Similar elements may be identified by a common reference character or numeral, with suffixes being used to refer to specific occurrences of the element

FIG. 1 shows a schematic block diagram of an example vision screening environment.

FIG. 2 shows a schematic block diagram of components of a vision screening device used in the visual acuity screening environment of FIG. 1.

FIGS. 3A-3D illustrates example light source configurations within the vision screening device shown in FIGS. 1 and 2.

FIG. 4 illustrate an example cross section of a human eye.

FIG. 5 shows an example of a schematic geometrical principle for measuring cornea curvature in accordance with examples of the present disclosure.

FIG. 6 is an example method associated with the example vision screening device shown in FIGS. 1 and 2.

FIG. 7 is another example method associated with the example vision screening device shown in FIGS. 1 and 2.

### DETAILED DESCRIPTION

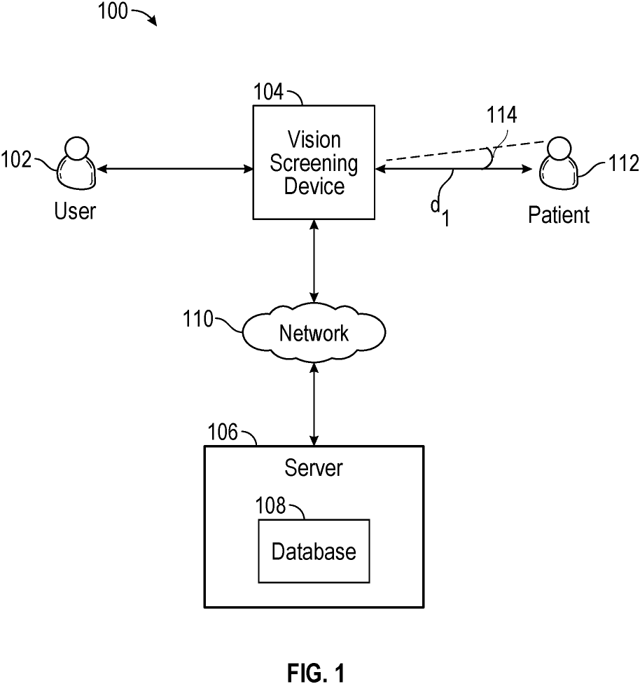

FIG. 1 is a schematic block diagram of an example visual acuity screening environment 100. The example visual acuity screening environment 100 includes a user 102, vision screening device 104, server 106, database 108, and a patient 112. Vision screening device 104 and server 106 are in communication via network 110. In typical operation, user 102 operates vision screening device 104 to test a patient 112 (e.g., any evaluated person). Other embodiments can include more or fewer components. For example, in any of the embodiments described herein, one or more of the refractive error determinations, cornea curvature determinations, axial length determinations, and/or other determinations may be made by a processor or other controller of the vision screening device 104, such as processing unit 208 described in greater detail below. In some examples, such determinations may be made by the processor or controller of the vision screening device 104 alone or at least partly in conjunction with the server 106.

Vision screening device 104 is a portable device configured to perform a vision screening test on the patient 112. Although common environments include schools and portable or permanent medical clinics, because vision screening device 104 is portable, it can be used virtually anywhere the user 102 takes the vision screening device 104. A commercial embodiment of example vision screening device 200 is the Spot™ Vision Screener VS100 by Welch Allyn, Inc.® (Skaneateles Falls, NY). Other embodiments can include more or fewer components as those described herein.

Vision screening device 104 is capable of performing both refractive error testing and facilitating vision screening testing. At a broad level, refractive error testing includes displaying stimuli, detecting pupils, acquiring images of the pupils, and analyzing pupil image data to generate refractive error results. As described in greater detail below, in some examples, vision screening testing includes determining a distance d1 of the patient 112 from the vision screening device 104, determining a cornea curvature of at least one eye of the patient, determining a prescription for the patient, and/or displaying the prescription. As described in greater detail below, in further examples, vision screening testing includes determining a distance d1 of the patient 112 from the vision screening device 104, determining an angle (e.g., gaze angle) 114 of the vision screening device 104 relative to the patient 112, determining a cornea curvature of at least one eye of the patient, determining an axial length of at least one eye of the patient 112, generating a recommendation for the patient, and/or displaying the recommendation.

In some examples, vision screening device 104 communicates with server 106, such as via network 110. For instance, a processor of vision screening device 104 may determine the refractive error results based on the analysis of pupil image data as noted above. In some examples, refractive error results are determined based at least in part on demographics, sphere, cylinder, axis, pupillometry and/or other characteristics of the patient 112. In still further examples, refractive error results are determined based at least partly on the accommodation range, binocular gaze deviation, pupillary reaction to the “brightness” of the fixation target, and pre-existing eye or neurological conditions. Objective visual acuity data, such as optic kinetic nystagintis (OKN) data can also be used. In some instances, the server 106 may have access to one or more of these data, for example, by communicating with the database 108 and/or with an electronic health record/electronic medical record database via network 110. In such examples, the server 106 may provide such information to the processor of the vision screening device 104 such that the processor of the vision screening device 104 can determine the refractive error of the patient 112 based at least in part on such data. Additionally or alternatively, such information may be stored locally within a memory associated with and/or in communication with the vision screening device 104 (e.g., such as memory of the processing unit 208, described in greater detail below). The processor of the vision screening device 104 may transmit refractive error testing results to the server 106 via network 110. Server 106, alone or in combination with database 108, determines corresponding vision acuity data based on the refractive error data received from vision screening device 104. In some examples, the server determines cornea curvature, axial length, a prescription of the patient, and/or a recommendation. In this example, the server 106 transmits the corresponding vision acuity data, prescription, and/or recommendation to the processor of the vision screening device 104. The processor of the vision screening device 104 uses the corresponding acuity data to provide a vision screening test for the patient 112. In some examples, the server 106 determines corresponding vision acuity data associated with the patient 112 and transmits the corresponding vision acuity data to the processor of the vision screening device 104. In this example, the processor of the vision screening device 112 uses the vision acuity data to determine one or more of cornea curvature, axial length, a prescription of the patient, and/or a recommendation for the patient 112.

In alternative implementations, vision screening device 104 determines corresponding vision acuity data based on the refractive error data. In those implementations, vision screening device 104 may communicate with server 106 to check for updates to any correspondence data or algorithms but otherwise does not rely on server 106 and/or database 108 for determining refractive error or corresponding acuity data. Vision screening device 104 and methods of using vision screening device 104 are described in greater detail below. In some instances, vision screening device 104 can be in communication with user 102 specific devices, such as mobile phones, tablet computers, laptop computers, etc., to deliver or communicate results to those devices.

Server 106 communicates with vision screening device 104 to respond to queries, receive data, and communicate with database 108. Communication from vision screening device 104 occurs via network 110, where the communication can include requests for corresponding acuity data. Server 106 can act on these requests from vision screening device 104, determine one or more responses to those queries, and respond back to vision screening device 104. Server 106 accesses database 108 to complete transactions by a vision screening device 104. In some examples, server 106 includes one or more computing devices, such as computing device 202 described in greater detail below.

Database 108 comprises one or more database systems accessible by server 106 storing different types of information. In some examples, database 108 stores correlations and algorithms used to determine vision acuity data based on refractive error testing. In some examples, database 108 stores clinical data associated with one or more patient(s) 112. In some examples, database 108 resides on server 106. In other examples, database 108 resides on patient computing device(s) that are accessible by server 106 via a network 110.

Network 110 comprises any type of wireless network or other communication network known in the art. In some examples, the network 110 comprises a local area network (“LAN”), a WiFi direct network, wireless LAN (“WLAN”), a larger network such as a wide area network (“WAN”), cellular network connections, or a collection of networks, such as the Internet. Protocols for network communication, such as TCP/IP, 802.11a, b, g, n and/or ac, are used to implement the network 110. Although embodiments are described herein as using a network 110 such as the Internet, other distribution techniques may be implemented that transmit information via memory cards, flash memory, or other portable memory devices.

Accordingly, the vision screening device 104 described herein may implement keratometry into photorefraction, thereby improving accuracy of cornea curvature determinations. Additionally, the techniques described herein enable a portable vision screening device to determine axial length and generate recommendations based in part on the axial length. This enables greater accessibility to vision screening exams and provides recommendations for patients 112 regarding potentially identified vision problems (e.g., such as myopia).

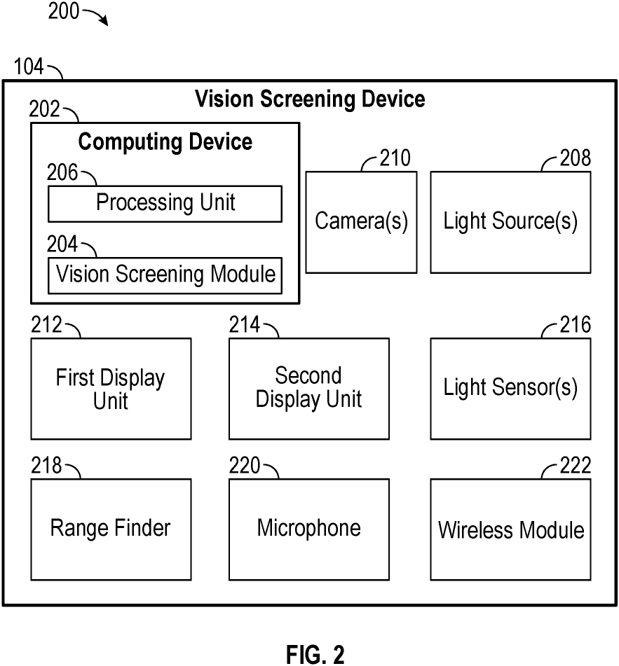

FIG. 2 is a schematic block diagram illustrating components of example vision screening device 104. As illustrated, example vision screening device 104 includes computing device 202, light source(s) 208, first display unit 212, second display unit 214, light sensor(s) 216, a range finder 218, a microphone 220, and a wireless module 222. In some examples, the vision screening device 104 comprises a housing (not shown), which provides support for components of vision screening device 104 as well as one or more aspects configured to facilitate hand-held operation. In some examples, one or more of the components of the vision screening device 104 are disposed within, partially disposed within, and/or are located on the housing.

Computing device 202 includes vision screening module 204 and processing unit 206. Vision screening module 204 comprises memory storing instructions for one or more of displaying a refractive error result on the first display unit 212, processing images received on the light source(s) 208, and guiding and informing the user 102 about optotype display and test results for the patient 112. Optotypes include, for example, letters, shapes, objects, and numbers. In some examples, the vision screening module is included as part of the processing unit 206 described below.

Processing unit 206 comprises one or more processor(s), controller(s), at least one central processing unit (“CPU”), memory, and a system bus that couples the memory to the CPU. In some examples, the memory of the processing unit 206 includes system memory and mass storage device. System memory includes random access memory (“RAM”) and read-only memory (“ROM”). In some examples, a basic input/output system (BIOS) that contains the basic routines that help to transfer information between elements within the example computing device 202, such as during startup, is stored in the ROM. In some examples, the mass storage device of the processing unit 206 stores software instructions and data. In some examples, mass storage device is connected to the CPU of the processing unit 206 through a mass storage controller (not shown) connected to the system bus. The processing unit 206 and its associated computer-readable data storage media provide non-volatile, non-transitory storage for the example computing device 202. Although the description of computer-readable data storage media contained herein refers to a mass storage device, such as a hard disk or solid state disk, it should be appreciated by those skilled in the art that computer-readable data storage media can be any available non-transitory, physical device or article of manufacture from which the central display station can read data and/or instructions.

Computer-readable data storage media include volatile and non-volatile, removable and non-removable media implemented in any method or technology for storage of information such as computer-readable software instructions, data structures, program modules or other data. Example types of computer-readable data storage media include, but are not limited to, RAM, ROM, EPROM, flash memory or other solid state memory technology, CD-ROMs, digital versatile discs (“DVDs”), other optical storage media, magnetic cassettes, magnetic tape, magnetic disk storage or other magnetic storage devices, or any other medium which can be used to store the desired information and which can be accessed by the example computing device 202.

In some examples, the processing unit 206 of the computing device 202 communicates with the components of vision screening device 104, including light source(s) 208, camera(s) 210, first display unit 212, second display unit 214, light sensor(s) 216, range finder 218, microphone 220, and wireless module 222. In some examples, vision screening device further comprises a lens (not shown), which may be adjustable. In this example, the processing unit 206 communicates with a controller of a device, such as a mechanical motor, that is configured to receive instructions from the processing unit 206 and, based at least partly on executing the instructions, adjust the position of the lens.

In some examples, the processing unit 206 is configured to instruct the light source(s) 208 and/or camera(s) 210 to capture image(s) of a cornea of a patient. In some examples and as described in greater detail below, the processing unit 206 is configured to generate an expected image of one or more expected locations of radiation returned from the cornea of a patient 112 based on a predetermined pattern of the light source(s) 208. The processing unit 206 is further configured to process and/or analyze images received via the light source(s) 208 and/or camera(s) 210 and determine, based at least partly on the image(s), one or more of refractive error, cornea curvature, axial length for one or more eyes of a patient 112. In some examples, the processing unit 206 is further configured to determine and/or generate a prescription for the patient or a recommendation for the patient 112. In some examples, the processing unit 112 is configured to display the prescription and/or recommendation on the first display unit 212. In some examples, the processing unit 206 processes and/or analyzes the image(s) using image processing techniques (e.g., positional analysis, object detection, etc.) and/or machine learning mechanisms.

Machine-learning mechanisms include, but are not limited to supervised learning algorithms (e.g., artificial neural networks, Bayesian statistics, support vector machines, decision trees, classifiers, k-nearest neighbor, etc.), unsupervised learning algorithms (e.g., artificial neural networks, association rule learning, hierarchical clustering, cluster analysis, etc.), semi-supervised learning algorithms, deep learning algorithms, etc.), statistical models, etc. In at least one example, machine-trained data models can be stored in memory associated with the computing device 202 and/or the server 106 for use during operation of the vision screening device 104.

Light source(s) 208 are configured to emit radiation (e.g., in the form of light) from the vision screening device 104 into an eye of a patient 112. In some examples, the light source(s) 208 comprise one or more light emitting diodes (LEDs), infrared (IR) LEDs, near IR LEDs, lasers (e.g., laser sensors), etc. In some examples, the light source(s) 208 comprise an LED array. In some examples, the LED array comprises visible LEDs, IR LEDs, and/or near-IR LEDs. In some examples, the near-IR LEDs in the LED array have a wavelength of about 850 nanometers (nm) and are used in capturing pupil images. Generally, the visible LEDs in the LED array have a wavelength of less than about 630 nm. This configuration allows for visual stimulus to be shown to the patient 112, but not seen in the images captured by the camera(s) 210 and/or light sensor(s) 216 described below. In some embodiments, the visible LEDs and/or IR LEDs are positioned between, and co-planar with, the near-IR LEDs in the LED array.

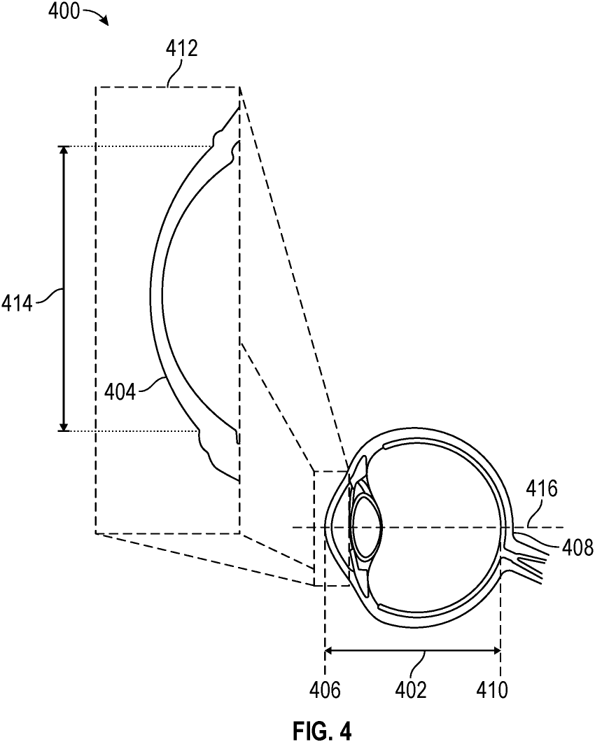

In some examples, the light source(s) 208 are configured in a predetermined pattern. For instance, as described in greater detail below with respect to FIG. 4, the predetermined pattern(s) comprise one or more of a star pattern, an array pattern, a placido ring pattern, a grid array pattern, a dot matrix pattern, a spot array pattern, a diamond pattern, and/or a circular pattern. However, any pattern of light source(s) 208 may be used. In some examples, the light source(s) 208 may be configured according to a custom pattern. In some examples, radiation is emitted from the light source(s) 208 comprise an array of IR LEDs configured in a predetermined pattern. In this example, the locations of each IR LED in the LED array is optimized, such that the camera(s) 210 and/or light sensor(s) 216 of the vision screening device 104 are enabled to capture clearer and/or sharper images of returned radiation. In some examples, the expected return location(s) (e.g., and/or expected pixel location(s)) are determined based at least partly on the configuration of the light source(s) 208. For instance, a location (e.g., within a predetermined pattern) of each light source 208 relative to the patient’s 112 eye is known and stored in memory of the processing unit 206 and/or other components of the vision computing device 104. The processing unit 206 can use the known locations of the light source(s) 208 and the predetermined pattern, to generate an expected return image that includes expected return locations of returned radiation based on the predetermined pattern. In some examples, additional information such as angle 114 of the vision screening device 104 relative to the patient 112 and/or distance (e.g., such as distance d1 described in FIG. 1 above) between the vision screening device 104 and the patient 112 is also used to determine expected return locations. As noted above, the processing unit 206 determines whether there is a difference between location(s) of returned radiation and expected return location(s) based at least partly on the expected image. The processing unit 206 determines the cornea curvature based at least partly on the difference between the returned location(s) and the expected return location(s). In some examples, the difference indicates whether there is significant asphericity of the patient’s eye and/or whether the patient 112 has an astigmatism. In some examples, the cornea curvature is determined using algorithms (examples of which will be described in greater detail below), look-up tables neural networks, machine learning models, or other processes. In this way, the vision screening device 104 utilizes light source(s) (e.g., IR LEDs) 208 in order to implement keratometry into photorefraction, thereby improving accuracy of cornea curvature determinations.

As illustrated, vision screening device 104 comprises one or more camera(s) 210. In some examples, the camera(s) 210 are configured to capture digital images of the patient’s eye and/or cornea in response to receiving instructions from the processing unit 206 and/or sensing returned radiation (e.g., such as via light sensor(s) 216, described below). For instance, in some examples, the camera(s) 210 comprise an image sensor array, such as a complementary metal-oxide semiconductor (CMOS) sensor array, also known as an active pixel sensor (APS), or a charge coupled device (CCD) sensor. In some examples, the camera(s) 210 comprise a lens that is supported by the vision screening device 104 and positioned in front of the light sensor array. The digital images are captured in various formats, such as JPEG, BITMAP, TIFF, PGM, PGV, etc. In some examples, the camera(s) 210 are configured to have a plurality of rows of pixels and a plurality of columns of pixels. In some embodiments, the camera(s) 210 comprise about 1280 by 1024 pixels, about 640 by 480 pixels, about 1500 by 1152 pixels, about 2048 by 1536 pixels, or about 2560 by 1920 pixels. In some examples, the camera(s) 210 are configured to capture about 25 frames per second (fps); about 30 fps; about 35 fps; about 40 fps; about 50 fps; about 75 fps; about 100 fps; about 150 fps; about 200 fps; about 225 fps; or about 250 fps. It is understood that the above pixel counts are merely examples, and in additional embodiments the light source(s) 208 may have a plurality of rows including greater than or less than the number of pixels noted above.

First display unit 212 conveys information to user 102 about the positioning of the vision screening device 104, including test results, recommendation(s), and/or prescription(s). In some examples, the first display unit 212 is positioned on a first end of the housing of the vision screening device 104, such that first display unit 212 faces the patient 112 during typical operation. In some examples, the first display unit 212 comprises a liquid crystal display (LCD) or active matrix organic light emitting display (AMOLED). In some examples, the first display unit 212 is touch-sensitive and configured to receive input from the user 102. Information provided to the user 102 via first display unit 212 comprises the patient’s 112 distance (e.g., such as distance d1 described in FIG. 1 above) from the vision screening device 104, a quality of the focus, progress of the evaluation, results of the evaluation, recommendations, prescription(s), and/or options for transmitting the results to another database (e.g., such as database 108 or any other database), via network 110.

Second display unit 214 displays one or more visual tests to the patient 112. In one implementation, second display unit 214 is a display, such as a liquid crystal display (LCD) or an active matrix organic light emitting display (AMOLED). As described above, the second display unit 214 communicates with computing device 202, via processing unit 206. In some examples, the second display unit 214 comprises one or more of the light source(s) 208 described above, such as a light-emitting diode (LED) array having visible LEDs, IR LEDs, and/or near-IR LEDs. In some examples, second display unit 214 is positioned on an opposite end of the housing of the vision screening device 104, relative to the first display unit 212, such that second display unit 214 faces the patient 112 during typical operation. In some examples, the second display unit 214 includes a display and one or more light source(s) 208 (e.g., LEDs or LED arrays). In some examples, the second display unit 214 comprises one or more of the light source(s) 208 described above, such as a light-emitting diode (LED) array having visible LEDs, IR LEDs, and/or near-IR LEDs. In some examples, the second display unit 214 comprises one or more amber LEDs in an LED array. Amber LEDs have a wavelength of about 608 nm to about 628 nm. The processing unit 206 regulates the amount of power directed to the LEDs in the LED array. For instance, in order to minimize the patient’s 112 pupil constriction and eye strain, the processing unit 206 instructs the second display unit 214 to emit radiation from the amber LEDs at low to medium power. For example, a 20 mA LED can be run at between about 2 mA to about 10 mA. Alternatively, low brightness amber LEDs can be used, for example, LEDs that run at about 0.5 mA. Additionally, LEDs can be pulse modulated. Visible light LEDs in colors other than amber, when present in the second display unit 214, can also be operated at low to medium power. Further, in some examples the vision screening device 104 may include one or more diffusers disposed in an optical path of one or more LEDs in the LED array. For example, such a diffuser may comprise a window, lens, prism, filter, and/or other substantially transparent optical component configured to at least partly diffuse radiation emitted by the one or more LEDs. As a result, for example, light emitted (e.g., as radiation) from the light source(s) 208 (e.g., by the one or more LEDs) of the second display unit 214 may not appear to be as bright when observed by the patient 112. In some such examples, diffusing light emitted by one or more of the LEDs in this way may reduce an amount of accommodation by the patient 112 and, as a result, the improve the accuracy of the refractive error measurement made by the vision screening device 104.

Light sensor(s) 216 of the vision screening device 104 comprise one or more sensor(s) configured receive light and conveys image data to processing unit 206 of computing device 202. In some examples, the light sensor(s) 216 comprise an image sensor array, such as a complementary metal-oxide semiconductor (CMOS) sensor array, also known as an active pixel sensor (APS), or a charge coupled device (CCD) sensor.

In some examples, a lens is supported by the vision screening device 104 and positioned in front of the light sensor(s) 216. For instance, in some examples, the light sensor(s) 216 are included as part of the camera(s) 210 described above. As noted above, in some examples, the light sensor(s) 216 are positioned on the interior of (e.g., disposed within) the housing of the vision screening device 104 and behind the second display unit 214, or adjacent thereto. Alternatively, the light sensor(s) 216 are positioned adjacent to second display unit 214 (e.g., below or above the second display unit 214) such that returned radiation need not pass through second display unit 214 to reach the light sensor(s) 216. Based at least in part on the returned radiation detected and/or sensed by the light sensor(s) 216, the camera(s) 210 capture one or more images of the cornea of the patient 112. In still further examples, the second display unit 214 may be disposed orthogonal to the light sensor(s) 216. In such examples, the second display unit 214 is configured to project an image onto a window, mirror, lens, or other substantially transparent substrate through which the light sensor(s) 216 detect the returned radiation.

In some examples, light sensor(s) 216 include photodiodes that have a light-receiving surface and have substantially uniform length and width. During exposure, the photodiodes convert the incident light to a charge. In some examples, the light sensor(s) 216 can be operated as a global shutter, that is, substantially all of the photodiodes are exposed simultaneously and for substantially identical lengths of time. Alternatively, the light sensor(s) 216 may be used with a rolling shutter mechanism, in which exposures move as a wave from one side of an image to the other. Other mechanisms are possible to operate the light sensor(s) 216 in yet other embodiments. In some examples, light sensor(s) 216 are capable of capturing digital images in response to receiving instructions from the processing unit 206. The digital images can be captured in various formats, such as JPEG, BITMAP, TIFF, PGM, PGV, etc.

In some examples, the light source(s) 208 and/or other components of the vision screening device 104 may perform one or more of the same functions (either alone or in combination with the light sensor(s) 216) described above with respect to the light sensor(s) 216. In particular, in some examples the light source(s) 208 may capture an initial image of the ambient surroundings. The computing device 202 may then determine, based at least in part on the captured image, whether there is too much ambient or IR light to perform one or more of the photorefraction operations described herein. If so, the computing device 202 may control the second display unit 214 to instruct the user 102 or patient 112 to use a light block, or move to an environment with less ambient light.

For example, in some embodiments the light source(s) 208 and/or the vision screening device 104, generally, may be configured to tolerate up to a threshold level of ambient IR light. In such examples, too much IR light from incandescent bulbs or sunlight may cause pupil images to be over exposed and washed out. Too much ambient visible light, by contrast, may cause the pupils of the patient 112 to be too small to measure with accuracy. In such examples, the light source(s) 208 and/or the vision screening device 104, generally, may be configured to sense both ambient visible and IR light, and to inform the user 102 as to visible and IR light levels that may be above respective thresholds. In such examples, a photodiode could be used to sense the overall level of ambient light, and an image captured by the light source(s) 208 with all the IR LED’s turned off could be used as a measure of ambient IR light.

In some examples, light sensor(s) 216 are configured to detect and/or sense information about the environment. For example, light sensor(s) 216 of vision screening device 104 may record the quantity of ambient light, time of day, ambient noise level, etc. This data can additionally be used to, for example, evaluate refractive error testing.

In some examples, light sensor(s) 216 detect the ambient light intensity around the vision screening device 104. Above certain brightness thresholds, the patient’s 112 pupils constrict to the point where the diameter of the pupil is so small that the vision screening device 104 may not be configured to determine the refractive error of the patient 112 accurately. If computing device 202, in combination with light sensor(s) 216, determines the ambient light is too bright, second display unit 214 communicates to the user 102 or patient 112 to use a light block or move to an environment with less ambient light. In some examples, the computing device 202 may also be configured to adjust and/or otherwise control the brightness, sharpness, contrast, and/or other operational characteristic of the second display unit 214 based at least in part on one or more signals received from the light sensor(s) 216. For example, based at least in part on the ambient light intensity measured by the light sensor(s) 216, the computing device 202 may be configured to adjust (e.g., automatically, dynamically, and/or in real time) the brightness, backlight, and/or other parameters of the second display unit 214 in order to maintain the contrast ratio at a desired level or within a desired range.

In some examples, the light source(s) 208 and/or other components of the vision screening device 104 may perform one or more of the same functions (either alone or in combination with the light sensor(s) 216) described above with respect to the light sensor(s) 216. In particular, in some examples the light source(s) 208 may capture an initial image of the ambient surroundings. The processing unit 206 of the computing device 202 may then determine, based at least in part on the captured image, whether there is too much ambient or IR light to perform one or more of the photorefraction operations described herein. If so, the processing unit 206 may control the second display unit 214 to instruct the user 102 or patient 112 to use a light block, or move to an environment with less ambient light.

For example, in some embodiments the light source(s) 208 and/or the vision screening device 104, generally, may be configured to tolerate up to a threshold level of ambient IR light. In such examples, too much IR light from incandescent bulbs or sunlight may cause pupil images to be over exposed and washed out Too much ambient visible light, by contrast, may cause the patient’s 112 pupils to be too small to measure with accuracy. In such examples, the light source(s) 208 and/or the vision screening device 104, generally, may be configured to sense both ambient visible and IR light, and to inform the user 102 as to visible and IR light levels that may be above respective thresholds. In such examples, a photodiode could be used to sense the overall level of ambient light, and an image captured by the light source(s) 208 with all the IR LED’s turned off could be used as a measure of ambient IR light.

Range finder 218, in combination with the processing unit 206 of the computing device 202, determines a distance (e.g., such as distance d1 described in FIG. 1 above) of the patient 112 from the vision screening device 104. In some examples, range finder 218 comprises an infrared transceiver unit, an ultrasonic transceiver unit, or another distance measuring unit known to one of skill in the art. Generally, the patient 112 is positioned about 1 meter (m), 10 feet, or 20 feet from the vision screening device 104. Other distances are possible, such as 16 inches, 20 inches, 30 inches, 35 inches, 40 inches, and 45 inches away. It is understood that the distances listed above are merely examples, and in additional embodiments, distances greater than or less than those noted above may be used during a visual acuity test and/or other tests described herein. As described above, the vision screening device 104 displays guidance to the patient 112 and/or the user 102 about how to adjust the relative positioning between the vision screening device 104 and the patient 112 to obtain a focal distance that will yield functional images. In embodiments where a user 102 operates the vision screening device 104, the guidance is displayed on first display unit 212. For example, first display unit 212 can display instructions to the user 102 indicating that the patient 112 is too close, too far away, or within a proper distance. In some embodiments, the focal length is about, 0.2 m, about 0.3 m, about 0.4 m, 0.5 m, about 0.6 m, about 0.7 m, about 0.75 m, about 0.8 m, about 0.9 m, about 1.0 m.

Microphone 220 senses audible sound and/or sound waved in inaudible frequencies. In some examples, the microphone 220 senses responses spoken by patient 112. In embodiments, the patient 112 speaks as part of the visual acuity test. For example, the patient 112 is asked to read an optotype, such as a letter, shown on the second display unit 214 and microphone 220 senses the patient’s 112 responses. Then computing device 202, in combination with voice recognition software, decodes the responses and uses the decoded responses in the visual acuity determination. Additionally, or alternatively, the user 102 may record the patient’s 112 responses manually and/or by interacting with one or more data input/touch input fields presented on the first display unit 212.

Wireless module 222 connects to external databases to receive and send refractive error and/or visual acuity test data using wireless connections. Wireless connections can include cellular network connections and connections made using protocols such as 802.11a, b, g, and/or ac. In other examples, a wireless connection can be accomplished directly between the vision screening device 104 and an external display using one or more wired or wireless protocols, such as Bluetooth, Wi-Fi Direct, radio-frequency identification (RFID), or Zigbee. Other configurations are possible. The communication of data to an external database can enable report printing or further assessment of the patient’s 112 test data. For example, data collected and corresponding test results are wirelessly transmitted and stored in a remote database accessible by authorized medical professionals.

Moreover, as noted above, the camera(s) 210 and/or light sensor(s) 216 capture one or more images of returned radiation from the patient’s 112 pupils. In some examples, the light source(s) 208 are configured in a predetermined pattern. The processing unit 206 of the computing device 202 and/or other components of the vision screening device 104 determine the patient’s 112 refractive error. In some examples, the refractive error may be determined based at least partly on information related to the sphere, cylinder, axis, gaze angle 114, pupil diameter, inter-pupillary distance, and/or other characteristics of the patient 112. The processing unit 206 of the computing device 202 and/or other components of the vision screening device 104 determine the patient’s cornea curvature based at least partly on the image(s). In some examples, the cornea curvature is determined based at least partly on the refractive error. In some examples and described in greater detail below, the computing device 202 and/or other components of the vision screening device 104 may utilize additional information in determining the patient’s cornea curvature. As described in greater detail below, the processing unit 206 and/or other components of the vision screening device 104 determine an axial length of the patient 112, based at least partly on the cornea curvature. In some examples, other characteristics (e.g., age, ethnicity, etc.) of the patient 112 are used to determine axial length. In some examples, the processing unit 206 and/or other components of the vision screening device 104 determine a prescription for the patient 112 based at least partly on the cornea curvature. In some examples, the processing unit 206 and/or other components of the vision screening device 104 generate a recommendation for the patient 112 based at least partly on the axial length.

Accordingly, the techniques herein enable a portable vision screening device to determine axial length and generate recommendations based in part on the axial length This enables greater accessibility to vision screening exams and provides recommendations for patients 112 regarding potentially identified vision problems (e.g., such as myopia).

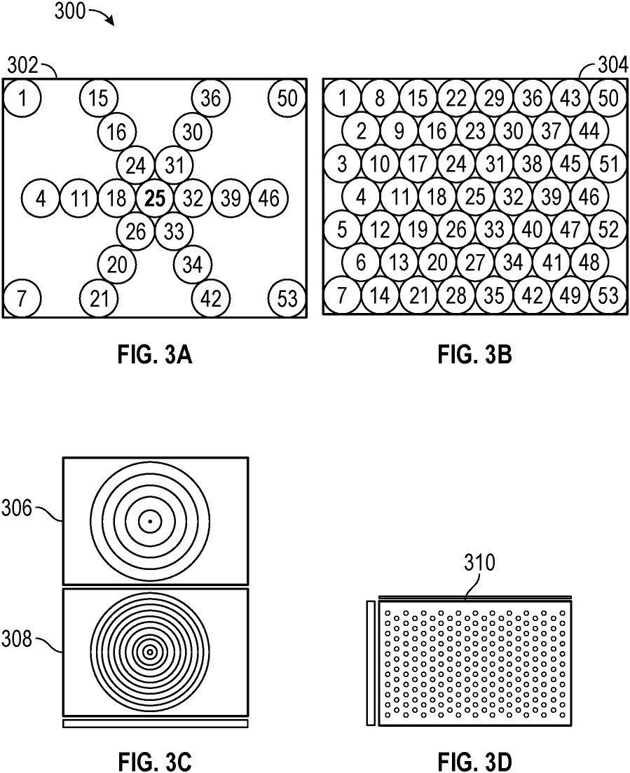

FIGS. 3A-3D illustrate examples of light source configurations within the example vision screening device 104 shown in FIGS. 1 and 2. In some examples, one or more of a lens resolution, image pixel size and count, and/or light source 208 locations are optimized to capture images with reflected radiation that are used to calculate cornea curvature. In this way, the patterns described below enable more accurate determinations of cornea curvature, thereby providing accurate prescriptions to patients 112. While FIGS. 3A-3D illustrate example configurations of predetermined light source patterns, any suitable pattern may be used (e.g., diamond pattern, circular pattern, grid array, 2-D patterns, among other patterns).

As illustrated in FIG. 3A, in some examples, the light source(s) 208 are configured according to a star pattern 302 and include light source(s) 208 at each corner (e.g., items 1, 7, 50, and 53). As described above, in some examples, one or more of the light source(s) 208 comprise visible LEDs, IR LEDs, lasers, and/or near-IR LEDs. In some examples, item 25 represents a light source 208 located in a central of the star pattern 302. In some examples, the center light source 25 continuously emits radiation during a visual screening exam. In this example, one or more of the other light sources 208 in the star pattern 302 also emit radiation during the visual screening exam. In some examples, one or more images of the patient’s 112 eye is captured during the visual screening exam. In this example, the light source(s) 208 emit radiation (e.g., in the form of light) towards the cornea of the patient 112 in the star pattern 302. Returned radiation that is reflected off of the cornea is captured by the camera(s) 210 and/or light sensor(s) 216 of the vision screening device 104 in one or more images. The processing unit 206 of the vision screening device 104 determines, based at least partly on the image(s), one or more locations of the returned radiation.

For instance, returned radiation from the center light source 25 may be associated with a particular pixel location in the image(s). The processing unit 206 determines whether there is a difference between the location(s) of the returned radiation and one or more expected locations of an expected image. For instance, the processing unit 206 determines whether there is a difference between the pixel location identified for the center light source 25 and an expected pixel location for the center light source 25. In some examples, the expected return location(s) (e.g., and/or expected pixel location(s)) are determined based at least partly on the configuration of the light source(s) 208. For instance, when the light source(s) 208 comprise a star pattern 302, a location of each light source 208 relative to the patient’s 112 eye is known and stored in memory of the processing unit 206 and/or other components of the vision computing device 104. The processing unit 206 can use the known locations of the light source(s) 208 and the predetermined pattern (e.g., star pattern 302), to generate an expected return image that includes expected return locations of returned radiation based on the star pattern. In some examples, additional information such as angle 114 of the vision screening device 104 relative to the patient 112 and/or distance between the vision screening device 104 and the patient is also used to determine expected return locations. As noted above, the processing unit 206 determines whether there is a difference between the pixel location identified for the center light source 25 and an expected pixel location for the center light source 25. For instance, the expected return pixel location for the center light source 25 may be a first point. However, the actual return location for the center light source 25 may be 3 pixels to the right of the first point and 1 pixel up from the first point. The processing unit 206 determines the cornea curvature based at least partly on the difference between the returned location(s) and the expected return location(s). In some examples, the difference indicates whether there is significant asphericity of the eye of the patient and/or whether the patient 112 has an astigmatism. In some examples, the cornea curvature is determined using the algorithms described in greater detail below. In this way, the vision screening device 104 utilizes light source(s) (e.g., IR LEDs) in order to implement keratometry into photorefraction, thereby improving accuracy of cornea curvature determinations.

As illustrated in FIG. 3B, in some examples, the light source(s) 208 are configured according to an array pattern 304. In some examples, the array pattern 304 comprises one or more IR LEDs. In some examples, item 25 represents a light source 208 located in a central of the array pattern 304. In some examples, the center light source 25 emits radiation during a visual screening exam. The light source 25 may emit radiation continuously, intermittently during the visual screening exam. In this example, one or more of the other light sources 208 in the array pattern 304 also emit radiation during the visual screening exam. In some examples, one or more images of the eye of the patient 112 is captured during the visual screening exam. As described above, the location of each light source(s) 208 in the predetermined pattern (e.g. array pattern 304) relative to the patient 112 is known and stored in memory of the processing unit 206 and/or other components of the vision screening device 104. The processing unit 206 can use the known locations of the light source(s) 208 and the predetermined pattern (e.g., array pattern 304), to generate an expected return image that includes expected return locations of returned radiation based on the array pattern 304. As descried above, additional information, such as a gaze angle 114 of the vision screening device 104 relative to the patient 112 and/or distance between the vision screening device 104 and the patient 112 is also used to determine expected return locations. As noted above, the processing unit 206 determines whether there is a difference between the pixel location identified for the center light source 25 and an expected pixel location for the center light source 25. For instance, the expected return pixel location for the center light source 25 may be a first point. However, the actual return location for the center light source 25 may be 3 pixels to the right of the first point and 1 pixel up from the first point. The processing unit 206 determines the cornea curvature based at least partly on the difference between the returned location(s) and the expected return location(s). In some examples, the difference indicates whether there is significant asphericity of the eye of the patient 112 and/or whether the patient 112 has an astigmatism. In some examples, the cornea curvature is determined using the algorithms described in greater detail below. The processing unit 206 determines based at least partly on the cornea curvature, a prescription for the patient 112. In this way, the vision screening device 104 utilizes light source(s) (e.g., IR LEDs) in order to implement keratometry into photorefraction, thereby improving accuracy of cornea curvature determinations.

As illustrated in FIG. 3C, in some examples, the light source(s) 208 are configured according to a first placido ring pattern 306 or a second placido ring pattern 308. In some examples, the placido ring patterns 306 and/or 308 are projected onto the cornea in order to capture a more complete measurement of the topology of the cornea. For instance, in some examples, rings of light sources 208, or an array of light sources 208 (such as an array of LEDs) and/or a mask (e.g., such as an optic light pipe or other mask configured to create particular pattern) to create the rings may be used. In some examples, the reflected radiation (in the form of rings) is captured by the camera(s) 210 and/or light sensor(s) 216 of the vision screening device 104. In some examples, the image(s) of the reflected radiation comprise information associated with the entire surface of the cornea, including where the rings (e.g., radiation) reflect from the outer surface of the cornea. As described above, the processing unit 206 determines whether there is a difference between one or more expected return location(s) of the returned radiation and one or more location(s) of the returned radiation. The processing unit 206 determines the cornea curvature based at least partly on the difference between the returned location(s) and the expected return location(s). In some examples, the difference indicates whether there is significant asphericity of the eye of the patient 112 and/or whether the patient 112 has an astigmatism. The processing unit 206 determines based at least partly on the cornea curvature, a prescription for the patient 112.

As illustrated in FIG. 3D, in some examples, the light source(s) 208 are configured according to a dot matrix pattern 310. In some examples, the dot matrix pattern 310 comprises a diffractive optical element, that is configured for use with a variety of different types of light source(s) 208 and/or wavelengths. For instance, in some examples, light source(s) 208 comprise lasers, LEDs, etc. In each example, the dot matrix pattern may comprise a molded diffractive element with the dot matrix pattern 310 that is placed in front of the light source(s) 208. In some examples, the dot matrix pattern 310 comprises a diffractive element, such as a beam splitter, configured to produce a spot array, such as a 25 x 5 spot array. As described above, the processing unit 206 determines whether there is a difference between one or more expected return location(s) of the returned radiation and one or more location(s) of the returned radiation. The processing unit 206 determines the cornea curvature based at least partly on the difference between the returned location(s) and the expected return location(s). In some examples, the difference indicates whether there is significant asphericity of the eye of the patient 112 and/or whether the patient 112 has an astigmatism. The processing unit 206 determines based at least partly on the cornea curvature, a prescription for the patient 112.

Accordingly, the vision screening device 104 may comprise light source(s) 208 configured in custom patterns that are optimized to capture image(s) of returned radiation from a patient’s 112 eye. The processing unit 206 of the vision screening device 104 determines cornea curvature based at least partly on difference(s) between location(s) of returned radiation and expected return location(s). Thus, the processing unit 206 of the vision screening device 104 can determine with a high accuracy cornea curvature of the patient and determine, based at least partly on the cornea curvature, a prescription for the patient.

In some examples, the processing unit 206 may additionally or alternatively generate a referral for the patient 112 based at least partly on the cornea curvature. In some examples, the referral is based at least partly on determining the patient has Keratoconus (e.g., a misshapen cornea). For instance, the processing unit 206 may determine whether the cornea curvature meets or exceeds a threshold. If the cornea curvature does meet or exceed the threshold, the processing unit 206 determines the cornea is misshapen and generates a referral for the patient 112. In some examples, the processing unit 206 determines the cornea is misshapen based at least partly on a refractive error, the image(s) of the eye of the patient, and/or other information accessible to the vision screening device 104 and/or server 106. In some examples, the processing unit 206 determines, based at least partly on the refractive error, image(s), and/or other information, whether there is a correlation between the eye of the patient and one or more symptoms of a misshapen cornea, and if so, the processing unit 206 may generate the referral.

FIG. 4 illustrates a cross section of a human eye 400 having an example axial length 402. As shown in FIG. 4, the axial length 402 is the distance from the cornea surface 404 at a front end 406 of the eye 400 to an interference peak corresponding to the retinal pigment epithelium and/or Bruch’s membrane 408 at a back end 410 of the eye 400. As described above, axial length may be used to (1) determine the risk of associated pathology for a patient 112, (2) predict risk for myopia development for the patient 112, and (3) evaluate the effectiveness of myopia management treatments. Accordingly, the vision screening device 104 determines recommendation for the patient based at least in part on an axial length 402 of a patient’s eye 400. In some examples, the recommendations may indicate whether the patient 112 should follow up with an eye doctor. For instance, in examples where large groups are being evaluated (e.g., such as at a school), the processing unit 206 of the vision screening device 104 displays the recommendations via the first display unit 212.

Moreover, FIG. 4 includes a partially exploded view 412, which includes an indication of cornea curvature 414. The cornea 404 is a tough, transparent membrane which forms the front surface of the eye. It represents the strongest part of the refracting power of the eye, providing about 80% of the power of the eye. As described above, cornea curvature is an indicator of whether the eye 400 of a patient 112 has an astigmatism, and can also guide a cornea refractive power correction surgery, and be used to determine a prescription for the patient 112 (e.g., such as a contact lens prescription, etc.).

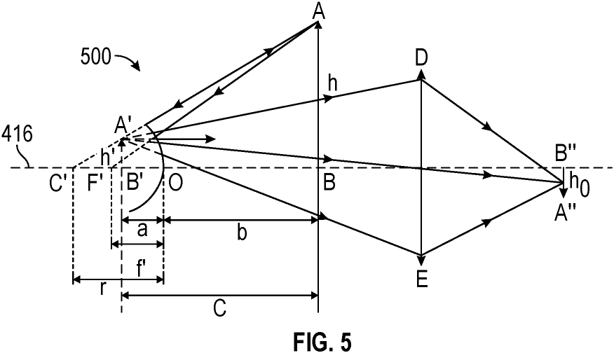

FIG. 4 further illustrates a central axis 416 of the eye 400. As described in greater detail below with regard to FIG. 5, the central axis 416 of the eye 400 can be used to determine cornea curvature 414. Accordingly, the techniques described herein enable a processing unit 206 of a portable vision screening device to determine a cornea curvature 414 of an eye 400 of a patient 112, axial length 402 of the eye 400, and generate recommendations, and/or prescriptions for the patient 112.

FIG. 5 shows an example of a schematic geometrical principle for measuring cornea curvature. As will be described below, the vision screening device of FIGS. 1 and 2 may be configured to use the geometric principle shown in FIG. 5 to make one or more of the determinations described herein. For instance, in some examples, the camera(s) 210 and/or the light sensor(s) 216 of the vision screening device 104 are used to capture image(s) of one or more eye(s) of the patient. In some examples, the camera(s) 210 and/or the light sensor(s) 216 of the vision screening device 104 are used to capture a binocular image (e.g., a single image of both eyes of the patient).

As indicated schematically in FIG. 5, a light source and/or a camera of the vision screening device may be placed at point A located a vertical distance is h away from the central axis 416 of an eye of the patient. A second distance, b, represents the distance from a vertex of the cornea of the eye to a light source of the vision screening device. As described above, the light source forms a virtual image at A′ and forms a digital image at A″. In some examples, the light source forms the digital image at A″, using a CMOS image sensor. As illustrated in FIG. 5, h₀ represents an image size of the digital image.

FIG. 5 further illustrates a cornea radius r, an optical system magnification β, a light source height h, and a virtual image height h′.

In some examples, one or more algorithms, data plots, graphs, lookup tables including empirical data, neural networks, and/or other items may be utilized by the vision screening device 104 to determine cornea radius, r. In such examples, the cornea radius, r, is determined using one or more algorithms such as:

$$r = \frac{2 b {h}_{0}}{β h - {h}_{0}}$$

and

$$β = \frac{{h}_{0}}{{h}^{′}}$$

In some examples, the vision screening device determines cornea curvature for both eyes of a patient based on a single image (e.g., a binocular image) taken by a camera of the vision screening device. In some examples, the binocular image may be captured using illumination of central light source(s) and/or eccentric illumination of the light source(s) described above. In this example, the vision screening device further utilizes an error analysis. In some examples, the error analysis determines an error, which represents a difference in a calculated value of cornea curvature radius between a camera of the vision screening device that is aligned with one eye of a patient and a camera of the vision screening device that is offset to the center of the patient’s two eyes. For instance, the vision screening device may receive input indicating characteristics of the patient (e.g., age and ethnicity). The vision screening device may then capture a binocular image of both eyes of the patient. The processing unit may perform one or more image processing techniques to determine cornea curvature, such as using techniques described above. Additionally or alternatively, the vision screening device may determine a cornea curvature further based on an offset value.

In some examples, one or more algorithms, data plots, graphs, lookup tables including empirical data, neural networks, and/or other items may be utilized by the vision screening device 104 to determine an offset value and an error. In such examples, the offset value and error are determined using one or more algorithms such as:

$${h}_{0} \text{} \_ \text{} o f f s e t = β ∗ \left(\frac{h ″ \text{} \_ \text{} U + h ″ \text{} \_ \text{} L}{2}\right)$$

and

$$E r r o r = \frac{2 b {h}_{0}}{β h - {h}_{0}} - \frac{2 b {h}_{0} \text{} \_ \text{} o f f s e t}{β \left(h + Δ h\right) - {h}_{0} \text{} \_ \text{} o f f s e t}$$

where the Δh represents an offset distance of the camera, and h″ represents a virtual image height of an offset LED. Additionally, 2 * h₀_offset is used to represent an image size of a full LED circle. As described above, b is the distance from a vertex of the cornea of the eye to the light source, h is a light source height, h₀ is an image size, h is a virtual image height, and β is an optical system magnification.

In some examples, the cornea curvature is used to determine an axial length of the eye 400, such as via a photographic method. For instance, the vision screening device may perform one or more image processing techniques on the binocular image to determine axial length for one or both eyes. In some examples, one or more algorithms, data plots, graphs, lookup tables including empirical data, neural networks, and/or other items may be utilized by the vision screening device 104 to determine the axial length. In some examples, axial length can be determined via the photographic method, using one or more algorithms such as:

$$A L = \frac{\left(\frac{n ∗ c s ∗ 0.5}{m a g}\right)}{\frac{L D}{W D}} ∗ 2 + r$$

where IIL is the axial length, n is the number of pixel separation on a sensor (e.g., such as sensor(s) used in camera(s) 210 and/or light sensor(s) 216 described above), cs is the cell size of the sensor unit, mag is the magnification of the camera 210, LD is the distance between the center of a light source (e.g., such as an LED) to the center of the camera, WD is a working distance of the camera 210, and r is the cornea radius.

Additionally, or alternatively, the cornea curvature is used to determine an axial length of the eye 400 and refractive error, based on data analysis. For instance, the vision screening device may perform one or more image processing techniques on the binocular image to determine axial length for one or both eyes. In some examples, one or more algorithms, data plots, graphs, lookup tables including empirical data, neural networks, and/or other items may be utilized by the vision screening device 104 to determine the axial length. In some examples, axial length can be determined via data analysis, using one or more algorithms such as:

$$\begin{matrix}A L = \left(\frac{24 ∗ A v e r a g e c o r n e a c u r v a t u r e}{7.8}\right) - \\ \left(s p h e r i c a l e q u i v a l e n t ∗ 0.4\right)\end{matrix}$$

In some examples, axial length of the eye 400 is determined using an improved formula aimed at minimizing error for each patient 112. For instance, the vision screening device axial length for one or both eyes of each patient 112 may be determined using the improved formula. In some examples, one or more algorithms, data plots, graphs, lookup tables including empirical data, neural networks, and/or other items may be utilized by the vision screening device 104 to determine the axial length. In some examples, axial length can be determined with minimized error, using one or more algorithms such as:

$$A L = {W}_{A} ∗ A {L}_{P G} + {W}_{B} ∗ A {L}_{D A} + C$$

where AL is the calibrated axial length result (e.g., improved result), AL_{PG} is the axial length determined using the photographic method described above, AL_{DA} is the axial length determined using data analysis described above, W_{A} is the weight of the photographic measurement, W_{B} is the weight of the data analysis prediction, and C is a compensation factor based on ametropia state. In some examples, AL_{DA} may be determined with a higher accuracy based on additional characteristics associated with the patient (e.g., patient age, patient ethnicity, etc.).

Accordingly, the techniques herein enable a portable vision screening device to determine axial length and generate recommendations based in part on the axial length for one eye, or both eyes (e.g., via the use of a binocular image). This enables greater accessibility to vision screening exams and provides recommendations for patients 112 regarding potentially identified vision problems (e.g., such as myopia).

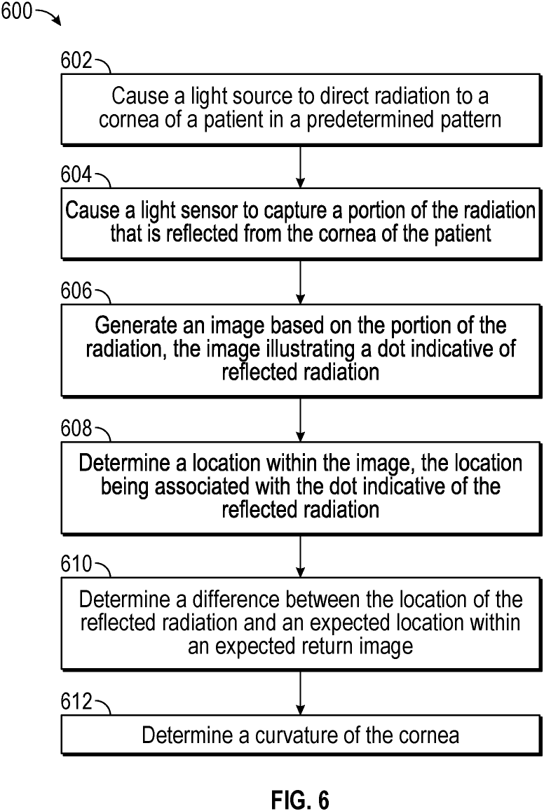

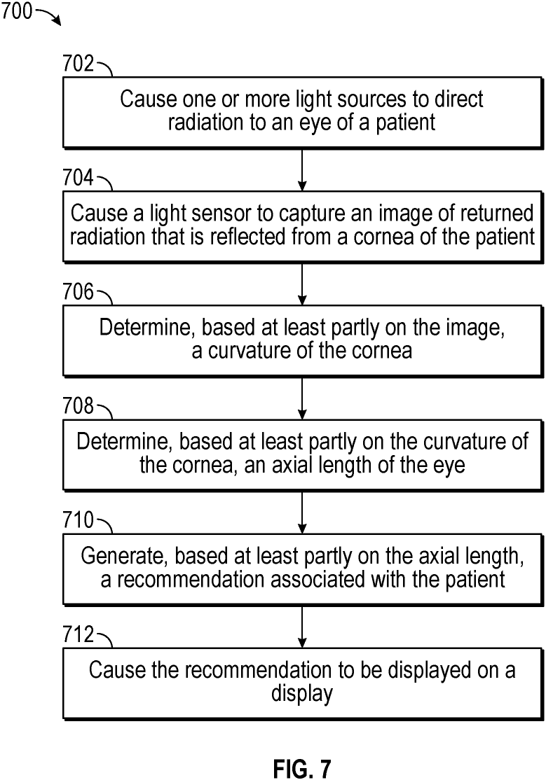

FIGS. 6 and 7 illustrate example methods 600, 700 associated with the vision screening device 104 shown in FIGS. 1 and 2. The example methods 600, 700 of FIGS. 6 and 7, respectively, are illustrated as logical flow graphs, each operation of which represents a sequence of operations that may be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described operations may be combined in any order and/or in parallel to implement the processes. Although any of the processes or other features described with respect to the methods 600 and/or 700 may be performed by processor(s) and/or controller(s) of server 106, for ease of description, the example methods 600 and 700 will be described below as being performed by the processor(s) 206 (e.g., processing unit 206 described above) of the vision screening device 104 unless otherwise noted.

As illustrated in FIG. 6, at 602, a processing unit 206 causes a light source 208 of a vision screening device 104 to direct radiation to a cornea of a patient 112 in a predetermined pattern. As described above, the light source 208 comprises one or more light emitting diodes (LEDs), infrared (IR) LEDs, near-IR LEDs, and/or laser sensors. As described above, the predetermined pattern comprises one of a star pattern, an array pattern, a grid array pattern, a diamond pattern, a circular pattern, a placido ring pattern, a dot matrix pattern, or a spot matrix pattern.

At 604, the processing unit 206 causes a light sensor to capture a portion of the radiation that is reflected from the cornea of the patient. In some examples, the processing unit 206 causes the camera(s) 210 and/or light sensor(s) 208 to capture the reflected radiation.

At 606, the processing unit 206 generates an image based on the portion of the radiation, the image illustrating a dot indicative of reflected radiation. In some examples, the image may be generated based on capturing the reflected radiation with a camera 210.

At 608, the processing unit 206 determines a location within the image, the location being associated with the dot indicative of the reflected radiation. In some examples and as described above, the processing unit 206 may analyze the image using various image processing techniques and/or machine learning mechanism(s) to determine location(s) of the returned radiation. For example, the processing unit 206 may analyze the image to identify one or more bright spot(s) in the image and may characterize the bright spot(s) as returned radiation.

At 610, the processing unit 206 determines a difference between the location of the reflected radiation and an expected location within an expected return image. As described above, in some examples, at 606 the processing unit 206 generates an image illustrating and/or otherwise illustrating an expected location of the returned radiation based at least partly on the predetermined pattern of the one or more light sources 208. In some examples, the expected location is associated with where the dot indicative of the reflected radiation is expected to be captured. In some examples, the expected location may be determined by the processing unit 206 using geometric methods (e.g., triangulation, etc.) associated with the testing conditions (e.g., distance between patient and the vision screening device 104, gaze angle, etc.) of the vision screening exam. Additionally or alternatively, the expected location may be determined based at least in part on a comparison to data stored in a lookup table that is associated with one or more predetermined conditions associated with the vision screening exam.

At 612, the processing unit 206 determines a curvature of the cornea (e.g., cornea curvature). As described above, the curvature of the cornea is determined based at least partly on the difference(s) between the location(s) of the reflected radiation and the expected location(s) in the expected image. For instance, as described above, In some examples, one or more algorithms, data plots, graphs, lookup tables including empirical data, neural networks, and/or other items may be utilized by the vision screening device 104 to determine corneal radius, r. In such examples, the corneal radius, r, is determined using one or more algorithms such as:

$$r = \frac{2 b {h}_{0}}{β h - {h}_{0}}$$

and

$$β = \frac{{h}_{0}}{{h}^{′}}$$

At 610, the processing unit 206 determines, based at least partly on the curvature, a prescription for the patient 112. As described above, the prescription may comprise a prescription for contact lenses. For instance, the processing unit 206 may access a database that contains prescriptions for contact lenses. In this example, may determine the prescription based at least in part on identifying a contact lens in the database that includes curvature value similar to the cornea curvature of the patient. The curvature value may be within a threshold amount. In some examples, the prescription may be based on whether the cornea curvature indicates that the patient has an astigmatism. For instance, the processing unit 206 may identify a prescription for contact lenses that are made for patients with astigmatisms. In some examples, the processing unit 206 displays the prescription on a display of the vision screening device 104, such as via the first display unit 212. In some examples, the process 206 sends the prescription to a computing device via a network 110, for display on the computing device.

Accordingly, the techniques herein enable a portable vision screening device to determine axial length and generate recommendations based in part on the axial length. This enables greater accessibility to vision screening exams and provides recommendations for patients 112 regarding potentially identified vision problems (e.g., such as myopia).

FIG. 7 is an example method associated with the example vision screening device shown in FIGS. 1 and 2. As illustrated in FIG. 7, at 702, a processing unit 206 causes one or more light sources 208 to direct radiation to an eye of a patient 112. In some examples, the processing unit 206 directs radiation from the one or more light sources into a second cornea the patient.

At 704, the processing unit 206 causes a light sensor to capture an image of returned radiation that is reflected from a cornea of the patient 112. In some examples, the processing unit 206 causes the camera(s) 210 and/or light sensor(s) 216 of the vision computing device 104 to capture the image. In some examples, the processing unit 206 causes the one or more camera(s) 210 and/or one or more light sensor(s) 216 to capture a second image of returned radiation that is reflected from the second cornea of the patient 112. In some examples, the image of returned radiation from the first cornea and the second image of returned radiation from the second cornea are different images captured at different times. In some examples, the processing unit 206 causes the camera(s) 210 and/or light sensor(s) 216 of the vision computing device 104 to capture an image (e.g., such as via a binocular image) of both the first cornea and the second cornea at a same time. For instance, in some examples, the image of the first cornea and the image of the second cornea comprise a same image (e.g., such as via a binocular image) that is captured at a same time by the vision screening device 104.

At 706, the processing unit 206 determines, based at least partly on the image, a curvature of the cornea. In some examples, the processing unit 206 determines, based at least partly on the second image a second curvature of the second cornea of the patient 112. In some examples, such as where the image and the second image comprise the same image (e.g., such that the image includes returned radiation from both the first cornea and the second cornea of the patient), at least one of the curvature of the first cornea or the second curvature of the second cornea is determined further based on a value associated with an error determination of an offset between the one or more cameras and a center of the eyes of the patient. In some examples, the error determination is based at least partly on one of the algorithms described above.

At 708, the processing unit 206 determines, based at least partly on the curvature of the cornea, an axial length of the eye. In some examples, the processing unit determines, based at least partly on the second curvature of the second cornea, a second axial length associated with a second eye of the patient 112. As described above, the second axial length may be determined at a same time as the axial length of eye and/or at a different time. In some examples, such as where the image and the second image comprise the same image (e.g., such as a binocular image where the image includes returned radiation from both the first cornea and the second cornea of the patient), the axial length of one or both eyes may be determined further based on a value associated with an error determination of an offset between the one or more cameras and a center of the eyes of the patient. In some examples, the error determination is based at least partly on one of the algorithms described above. In some examples, the processing unit 206 receives, via a display of the vision screening device 104, such as the second display unit 214, input indicating an age and/or ethnicity of the patient 112. In some examples and as described above, the axial length and/or second axial length is further based at least partly on a characteristic associated with the patient and a refractive error, the characteristic comprising one or more of age and ethnicity.

At 710, the processing unit 206 generates, based at least partly on the axial length, a recommendation associated with the patient 112. In some examples, the recommendation is further generated based at least partly on the second axial length of the second eye of the patient 112. As described above, in some examples, the recommendation comprises an indication of whether the patient requires a follow-up consultation, such as when myopia is identified.

At 712, the processing unit 206 causes the recommendation to be displayed on a display. In some examples, the processing unit 206 causes the recommendation to be displayed on a display, such as the first display unit 212, of the vision computing device 104. In some examples, the processing unit 206 sends the recommendation, via a network, to a computing device associated with a user for display. Accordingly, the techniques described herein provide a handheld and/or portable vision screening device that can capture image(s) of returned radiation, determine cornea curvature based in part on the images, determine an axial length of the eye, and generate recommendations based in part on the axial length.

As noted above, the example devices and systems of the present disclosure may be used to perform vision screening tests. For example, components described herein may be configured to utilize IR LEDs to capture reflected radiation according to predetermined and/or customized patterns, determine difference(s) between locations of the reflected radiation and where the radiation is expected to be captured, determine the curvature of a cornea based at least in part on the difference, and determine a prescription for a patient. Additionally, the components described herein may be configured to capture image(s) of returned radiation, determine cornea curvature based in part on the images, determine an axial length of the eye, and generate a recommendation based in part on the axial length.

As a result, the devices and systems described herein may assist a user with determining cornea curvature with improved accuracy and determining a prescription and/or referral for a patient, thereby streamlining vision screening exams. Moreover, the devices and systems described herein may assist a user with determining axial length and determining a recommendation for a patient, thereby providing an integrated vision screening exam and reducing time of the vision screening exams. Additionally, by enabling a portable and/or handheld vision screening device to perform the improved cornea curvature determination and the axial length determinations, the device and systems described herein enable the vision screening device to perform operations previously unavailable to patients via a portable device. This may streamline workflow for providing prescriptions, follow-up recommendations, and/or referrals for primary care physicians and others, thereby reduce the cost of treatments.

The foregoing is merely illustrative of the principles of this disclosure and various modifications can be made by those skilled in the art without departing from the scope of this disclosure. The above described examples are presented for purposes of illustration and not of limitation. The present disclosure also can take many forms other than those explicitly described herein. Accordingly, it is emphasized that this disclosure is not limited to the explicitly disclosed methods, systems, devices, and apparatuses, but is intended to include variations to and modifications thereof, which are within the spirit of the following claims.

As a further example, variations of apparatus or process limitations (e.g., dimensions, configurations, components, process step order, etc.) can be made to further optimize the provided structures, devices, and methods, as shown and described herein. In any event, the structures and devices, as well as the associated methods, described herein have many applications. Therefore, the disclosed subject matter should not be limited to any single example described herein, but rather should be construed in breadth and scope in accordance with the appended claims.

In some instances, one or more components may be referred to herein as “configured to,” “configurable to,” “operable/operative to,” “adapted,“adaptable,” “able to,” “conformable/ conformed to,” etc. Those skilled in the art will recognize that such terms (e.g., “configured to”) can generally encompass active-state components and/or inactive-state components and/or standby-state components, unless context requires otherwise.

The description and illustration of one or more embodiments provided in this application are not intended to limit or restrict the scope of the invention as claimed in any way. Regardless whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate embodiments falling within the spirit of the broader aspects of the claimed invention and the general inventive concept embodied in this application that do not depart from the broader scope.

What is claimed is:

1. A system, comprising:
a processing unit;
one or more light sources operatively connected to the processing unit;
a light sensor operatively connected to the processing unit; and
non-transitory computer-readable media storing instructions that, when executed by the processing unit, cause the processing unit to perform operations comprising:
causing the one or more light sources to direct radiation to a cornea of a patient in a predetermined pattern;
causing the light sensor to capture a portion of the radiation that is reflected from the cornea of the patient;
generating an image based on the portion of the radiation, the image illustrating a dot indicative of reflected radiation;
determining a location within the image, the location being associated with the dot indicative of the reflected radiation;
determining a difference between the location of the reflected radiation and an expected location within an expected return image, the expected location being associated with where the dot indicative of the reflected radiation is expected to be captured; and
determining, based at least in part on the difference, a curvature of the cornea.

2. The system of claim 1, wherein the one or more light sources are disposed within a housing of a vision screening device and arranged in a predetermined pattern, the predetermined pattern comprising one of a grid pattern, a diamond pattern, a circular pattern, a placido ring pattern, a dot matrix pattern, or a spot matrix pattern.

3. The system of claim 1, wherein the one or more light sources comprise one or more light emitting diodes (LEDs).

4. The system of claim 1, wherein the one or more expected locations associated with the expected return image are based at least partly on locations of the light sources and the predetermined pattern.

5. The system of claim 1, further comprising:
determining, based at least partly on the curvature of the cornea, a prescription for the patient; and
displaying the prescription on a display of a vision screening device.

6. The system of claim 1, further comprising:
generating the expected return image based at least partly on the predetermined pattern of the one or more light sources.

7. The system of claim 1, further comprising:
sending to a remote server, information associated with the patient, the information including a recommendation associated with the patient.

8. A vision screening device, comprising:
a processing unit;
a housing;
one or more light sources disposed within the housing and operatively connected to the processing unit,
a light sensor disposed within the housing and operatively connected to the processing unit; and
memory storing instructions that, when executed by the processing unit, cause the vision screening device to:
cause the one or more light sources to direct radiation to a cornea of a patient in a predetermined pattern;
cause the light sensor to capture a portion of the radiation that is reflected from the cornea of the patient;
generate an image based on the portion of the radiation, the image illustrating a dot indicative of reflected radiation;
determine a location within the image, the location being associated with the dot indicative of the reflected radiation;
determine a difference between the location of the reflected radiation and an expected location within an expected return image, the expected location being associated with where the dot indicative of the reflected radiation is expected to be captured; and
determine, based at least partly on the difference, a curvature of the cornea.

9. The vision screening device of claim 8, further comprising:
a display unit;
the memory further storing instructions that, when executed by the processing unit, cause the vision screening device to:
determine, based at least partly on the curvature of the cornea, a prescription for the patient; and
display the prescription on the display unit.

10. The vision screening device of claim 8, wherein the one or more light sources are configured to emit the radiation according to the predetermined pattern, the predetermined pattern comprising one of a grid pattern, a diamond pattern, a circular pattern, a placido ring pattern, a dot matrix pattern, or a spot matrix pattern.

11. The vision screening device of claim 8, further comprising a range finder, the range finder being used for determining a distance of the patient from the vision screening device.

12. The vision screening device of claim 8, the memory further storing instructions that, when executed by the processing unit, cause the vision screening device to:
generate the expected return image based at least partly on the predetermined pattern of the one or more light sources.

13. A system implemented by a vision screening device, the system comprising:
a processing unit;
one or more light sources operatively connected to the processing unit;
a light sensor operatively connected to the processing unit; and
one or more non-transitory computer-readable media storing instructions that, when executed by the processing unit, cause the processing unit to perform operations comprising:
causing the one or more light sources to direct radiation to a first cornea of an eye of a patient;
causing the light sensor to capture an image of returned radiation that is reflected from the first cornea of the patient;
determining, based at least partly on the image, a curvature of the first cornea,
determining, based at least partly on the curvature of the first cornea, an axial length associated with the eye, and
generating, based at least partly on the axial length, a recommendation associated with the patient.

14. The system of claim 13, further comprising:
cause the one or more light sources to direct radiation to a second cornea of the patient;
cause the light sensor to capture a second image of returned radiation that is reflected from the second cornea of the patient;
determining, based at least partly on the second image, a second curvature of the second cornea; and
determining, based at least partly on the second curvature of the second cornea, a second axial length,
wherein the recommendation is generated further based at least partly on the second axial length.

15. The system of claim 14, wherein the image is captured at a first time and the second image is captured at a second time, different from the first time.

16. The system of claim 14, wherein the image and the second image are captured at a same time and comprise a same image.

17. The system of claim 16, wherein at least one of the curvature of the first cornea or the second curvature of the second cornea is determined further based on a value associated with an error determination of an offset between the one or more cameras and a center of the eyes of the patient.

18. The system of claim 13, further comprising:
causing the recommendation to be displayed on a display of the vision screening device, wherein the recommendation comprises an indication of whether the patient requires a follow-up consultation.

19. The system of claim 13, wherein the recommendation comprises an indication of whether the patient requires a follow-up consultation, the operations further comprising:
sending, via a network, the recommendation to a computing device for display.

20. The system of claim 13, wherein the axial length is further based at least partly on a characteristic associated with the patient and a refractive error, the characteristic comprising one or more of age and ethnicity.
